(19) United States
(12) Patent Application Publication
(10) Pub. No.: US 2020/0289323 A1
(43) Pub. Date: Sep. 17, 2020

(54) A WELDING HELMET WITH A DETACHABLE WELDING VISOR

(71) Applicant: 3M INNOVATIVE PROPERTIES COMPANY, St. Paul, MN (US)

(72) Inventors: Björn Daniels, Vikarbyn (SE); Thomas B. Stenvall, Sundborn (SE)

(21) Appl. No.: 16/767,713

(22) PCT Filed: Dec. 6, 2018
(86) PCT No.: PCT/IB2018/059736

§ 371 Date: May 28, 2020

(30) Foreign Application Priority Data
Dec. 8, 2017 (EP) 17206073.3

(51) Int. Cl. A61F 9/06

(52) U.S. Cl. CPC A61F 9/064; A61F 9/067

(57) ABSTRACT

There is provided a welding helmet having a protective shield and a welding visor. The welding visor is detachably attachable to the protective shield by means of a bayonet coupling. The bayonet coupling has a first bayonet part and a second bayonet part. The first bayonet part is rotatably connected to the welding visor for a rotation between a release position, in which the bayonet coupling is disengaged, and an engage position, in which a disengagement of the bayonet coupling is disabled. The bayonet coupling further forms a pivot for pivoting the welding visor relative to the protective shield between a welding position, in which the welding visor covers the see-through window, and a non-welding position, in which the welding visor uncovers the see-through window. A locking member is provided that is switchable between a locked position and an unlocked position.

### FIELD

The present disclosure relates to welding helmet that has a protective shield and a welding visor that are detachably attachable to each other by means of a bayonet coupling. The bayonet coupling has a first bayonet part and a second bayonet part. The first bayonet part can be rotated between a release position, in which the bayonet coupling is disengaged, and an engage position, in which a disengagement of the bayonet coupling is disabled.

### BACKGROUND ART

Welding Helmets are typically used in the mechanical and industrial art to protect welders from harmful irradiation emitted from the welding arc and from splashes, sparks and particles that may be ejected from a welding area. Welding helmets typically can be suspended on the head of a wearer, so that the wearer has both hands available for welding and handling of workpieces.

Some welding helmets are furnished with an automatic darkening filter. An automatic darkening filter commonly has a switchable filter that automatically changes from a light-state to a dark-state in response to incident light generated by the welding arc. Thus, upon ignition of the welding arc the switchable filter automatically changes to the dark-state and protects the welder's eyes from the irradiation of the welding arc. Once the welding is interrupted or ended the switchable filter automatically changes to the light-state so that the user can see through the filter at normal light conditions.

Under certain circumstances a welder may not only weld a workpiece but may desire to carry out additional treatments. For example, the welder may grind the workpiece or perform cutting or other works at the workpiece.

Although a welding helmet with an automatic darkening filter can provide protection and visibility during additional treatments there is still a need for a welding helmet that can be used for different purposes including welding.

### SUMMARY

The present disclosure relates to a welding helmet. The welding helmet comprises a protective shield and a welding visor. The protective shield is suspended at a head suspension system. Further, the welding visor is detachably attachable to the protective shield by means of a bayonet coupling. The bayonet coupling comprises a first bayonet part and a second bayonet part. The first bayonet part is rotatably connected to the welding visor for a rotation between a release position and an engage position.

In the release position the bayonet coupling is disengaged and in the engage position a disengagement of the bayonet coupling is disabled.

The bayonet coupling further forms a pivot for pivoting the welding visor relative to the protective shield between a welding position and a non-welding position.

In the welding position the welding visor covers the see-through window and in the non-welding position the welding visor uncovers the see-through window.

A locking member is provided that is switchable between a locked position and an unlocked position.

In the locked position the rotation of the first bayonet part from the engage position toward the release position is blocked, and in the unlocked position the rotation of the first bayonet part from the engage position toward the release position is enabled.

The present disclosure is advantageous in that it provides for a welding helmet that is flexible in use. In particular, the welding helmet can be used for non-welding purposes, for example for grinding or for other treatments in which a face protection for a wearer of the welding helmet is recommendable. The welding helmet of the present disclosure further allows the welding visor to be temporarily entirely removed. Thus, the welding visor can be kept clean or can be protected against damages, for example when exposed to sparks from grinding or ink during spraying. Further the welding helmet of the present disclosure allows for easy and quick removing the welding visor from the protective shield and for easy and quick mounting of the welding visor to the protective shield. In addition, the welding visor can be locked against inadvertent removal from the protective shield.

In the release position the welding visor can be detached from the protective shield if the welding visor is present on the protective shield. Further, in the release position the welding visor can be attached on the protective shield if the welding visor is separated from the protective shield. This is because in the release position the bayonet coupling is disengaged and therefore allows for being separated or combined. It is noted that although the bayonet coupling may be disengaged the welding visor and the protective shield may be retained to each other via the disengaged but still merged bayonet coupling.

In some embodiments, the second bayonet part is formed by a rim that is connected to the protective shield. The rim preferably has a cylindrical shape. The first bayonet part is preferably formed by a ring-shaped collar for mating on the rim. The collar is preferably rotatably mounted in a through-hole of the welding visor. In particular, an outer surface of the collar may be cylindrical and guided by a cylindrical inner surface of the through-hole. Thereby the first bayonet part may be rotatably connected to the welding visor. The collar is preferably axially fixed or secured to the welding visor but rotatable. The term “axially” in this regard refers to a dimension along a rotation axis of the collar.

In some embodiments, the first bayonet part comprises a first engagement member. The first engagement member preferably protrudes radially inwardly from the collar. In particular, the collar may have an inner cylindrical surface from which the first engagement member protrudes. The second bayonet part preferably comprises a second engagement member that protrudes radially outwardly from the rim. The rim may have an outer cylindrical surface from which the second engagement member protrudes. The rim with the second engagement member preferably is shaped and sized for mating within the collar. The engagement of the bayonet coupling is preferably established by an engagement of the first and second engagement member, in particular when the first and second bayonet part are mated and positioned in a rotational position relative to each other in which the second engagement member axially restrains the first engagement member.

In some embodiments, the first bayonet part has a lever that protrudes radially outwardly from the collar. The lever enables a wearer to operate the collar, for example during wearing the welding helmet, for detaching and/or attaching the welding visor from or to the protective shield, respectively. The locking member is preferably displaceably arranged for a displacement between the locked position and the unlocked position. The locking member is preferably displaceably arranged at the lever. The displacement is preferably provided along a dimension in which the lever protrudes. Accordingly, the displacement is preferably enabled in only a radial dimension of the collar or the rotation axis of the collar. This helps avoiding inadvertent switching of the locking member when the welding visor is pivoted relative to the protective shield because the radial displaceability is transverse to the tangential movement of the welding visor.

In some embodiments, the welding visor adjacent the through hole comprises a receptacle. The locking member in the locked position preferably engages in the receptacle and thereby locks the first bayonet part and the welding visor with each other.

In some embodiments, the welding helmet further has a link for pivotally suspending the protective shield at the head suspension system. Accordingly, the link may suspend the protective shield at the head suspension system. The link preferably comprises a knob for adjusting a retention against pivoting of the protective shield relative to the head suspension system. The link may further comprise a screw nut and the knob may comprise a screw bolt for establishing a screw connection with the screw nut. The protective shield can be suspended at the head suspension system by establishing the screw connection with a portion of the protective shield and a portion of the head suspension system arranged between the knob and the screw nut. The welding helmet particularly may have two links (as described herein) that are arranged on opposite sides of the suspension system for pivotally suspending the protective shield at the head suspension system.

In some embodiments, the see-through window comprises a clear protective plate. For example, the clear protective plate may seal the see-through window. The clear protective plate may be flat or curved. Further, the clear protective plate may be made of a polycarbonate material, a polymethyl methacrylate or a safety glass.

In some embodiments, the welding visor comprises an Automatic Darkening Filter (ADF). The automatic darkening filter is preferably based on two liquid crystal cells. The liquid crystal cells are electrically switchable between a light-state and a dark-state. The two liquid crystal cells are preferably arranged optically in sequence. Each liquid crystal cell comprises two transparent substrates with a liquid crystal layer arranged between. Each substrate is provided with an alignment layer that is in contact with the liquid crystal layer. The alignment layers provide for a default uniform alignment of the liquid crystals. Further, the two liquid crystal cells preferably comprise three polarizers, one of which being arranged between the two liquid crystal cells and the other two being arranged on outer sides. The outer side polarizers may be arranged with their light polarizing orientation in the same or substantially the same direction, whereas the inner polarizer may be oriented with its light polarizing orientation 90 degrees or substantially 90 degrees relative to the outer polarizers.

In the light-state the transmittance of the automatic darkening filter may be within a range of about 1% to about 20%, in more particular within a range of about 5% to about 10%, whereas in the dark-state the transmittance of the automatic darkening filter may be within a range of about 0.0005% to about 0.1%.

In some embodiments, the welding visor comprises a permanent optical filter (instead of an automatic darkening filter). Such an optical filter may have a permanent transmittance within a range of about 0.0005% to about 0.1%.

In some embodiments, the welding visor comprises a static darkening filter. Static darkening filters are well known in the art. The static darkening filter comprises a filter that blocks ultraviolet and infrared light.

In some embodiments, the welding helmet has a sensor for detecting light, as for example light emitted from the welding arc. The sensor and the automatic darkening filter are functionally interconnected so that light above a predetermined light intensity detected by the sensor causes the automatic darkening filter to switch to the dark-state and the absence or non-detection of light above the predetermined light intensity causes the automatic darkening filter to switch to the light-state.

In some embodiments, in the welding position the welding visor seals an area on the protective shield in which the see-through window is located. This prevents dust or other undesired substances from penetrating between the darkening filter and the clear protective plate.

In some embodiments, the bayonet coupling in the engage position of the first bayonet part provides an engagement of the welding visor relative to the protective shield over the full pivot range between the welding visor and the protective shield. The pivot range between the welding visor and the protective shield is preferably at least 50 degrees, preferably between 50 degrees and 60 degrees, for example 51 degrees. Thus, the see-through window can be fully uncovered from the welding visor in the non-welding position.

In some embodiments, the second bayonet part is monolithically formed with the protective shield. For example, the protective shield may be injection molded including the second bayonet part.

In some embodiments, the welding visor is detachably attachable to the protective shield by means of two bayonet couplings that are arranged on opposite sides of the protective shield. Each bayonet coupling of these embodiments is formed of a first bayonet part and a second bayonet part as described herein. The two bayonet couplings in combination form a pivot for pivoting the welding visor relative to the protective shield between the welding position and the non-welding position. Each of the bayonet couplings has a locking member that is switchable between the locked position and the unlocked position as described herein.

### BRIEF DESCRIPTION OF THE FIGURES

FIG. 1 is a side view of a welding helmet in a welding position according to some embodiments of the present disclosure;

FIG. 2 is a side view of the welding helmet shown in FIG. 1 but in a non-welding position;

FIG. 3 is a perspective view of a welding helmet according to some embodiments of the present disclosure, having a welding visor and a protective shield that are detached from each other;

FIGS. 4 to 6 are partial side views illustrating an operation of a welding helmet according to some embodiments of the present disclosure;

FIG. 7 is a perspective view of a collar and a locking member for a welding helmet according to some embodiments of the present disclosure;

FIG. 8 is a perspective cross-sectional partial view of a welding helmet according to some embodiments of the present disclosure; and

FIG. 9 is a further perspective cross-sectional partial view of a welding helmet according to some embodiments of the present disclosure.

### DETAILED DESCRIPTION

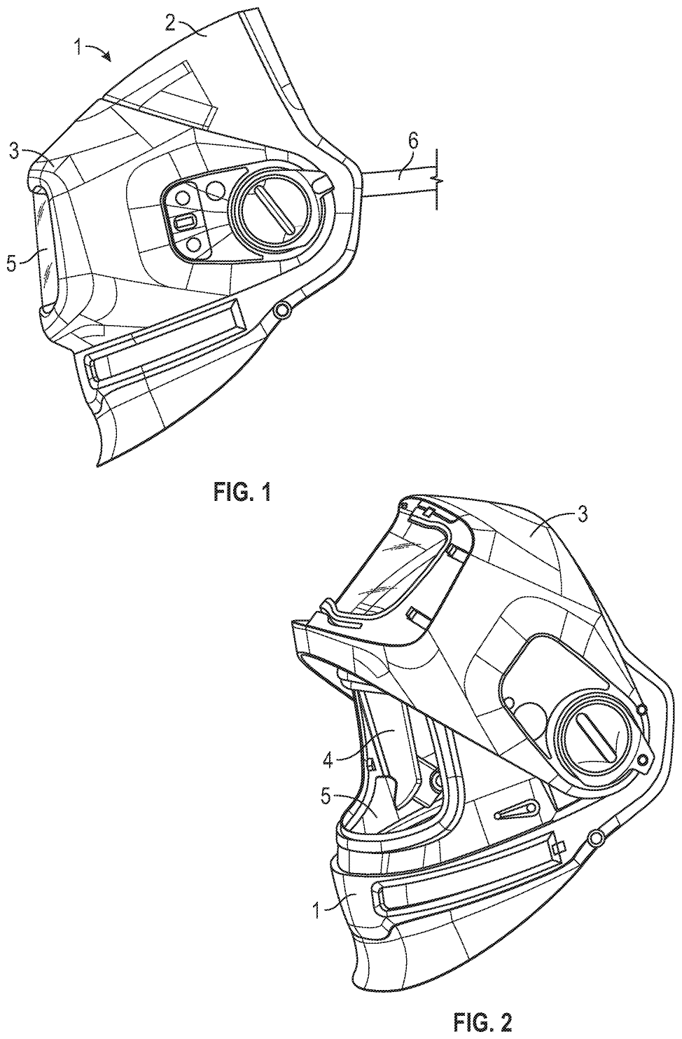

FIG. 1 shows a welding helmet 1 according to the present disclosure. The welding helmet 1 has a protective shield 2 which is illustrated in a lowered position that corresponds to a position in which the protective shield 2 covers a wearer's face. The welding helmet 1 further has a head suspension system 6 for supporting the welding helmet 1 on a wearer's head. The welding helmet 1 further has a welding visor 3. The welding visor 3 is configured to filter those portions of light of a welding arc hat would be harmful for a wearer of the welding helmet 1 observing the welding arc through the welding visor 3.

The welding visor 3 therefore comprises an automatic darkening filter 5. The automatic darkening filter 5 allows a welder to safely observe the welding arc during welding. In the example the automatic darkening filter 5 is based on two liquid crystal cells. The liquid crystal cells are electrically switchable between a light-state and a dark-state. When switched in the dark-state, the automatic darkening filter 5 blocks a significant amount of light from being transmitted therethrough. This enables a user to observe a welding arc by seeing through the automatic darkening filter 5 without risking exposure to harmful light radiation from the welding arc. In the light-state the automatic darkening filter 5 permits a significant amount of light to be transmitted therethrough. Thus, the automatic darkening filter 5 in the light-state allows the user to see under ambient light conditions (in the absence of the welding arc). The two (or more) liquid crystal cells are arranged optically in sequence. This provides for multiplying the darkening effect (in particular in the dark-state) and thus a sufficient eye protection from light radiation.

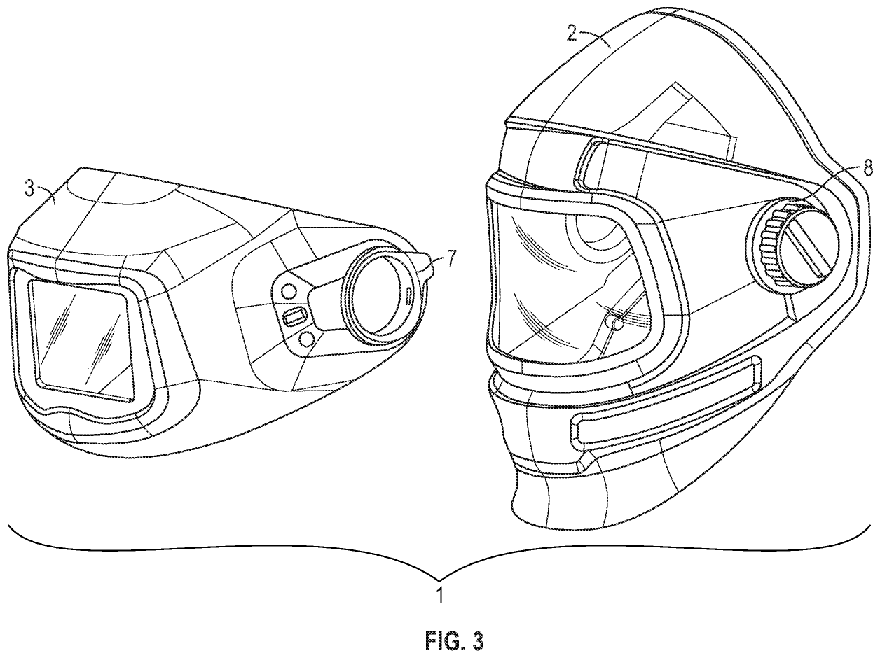

The welding visor 3 is illustrated in a welding position in which the welding visor 3 covers a see-through window (indicated as number 4 in FIG. 2) provided in the protective shield 2. The welding visor 3 is however pivotable between the welding position (shown in FIG. 1) and a non-welding position (shown in FIG. 2).

FIG. 2 shows the welding helmet 1 with the welding visor 3 in the non-welding position in which the welding visor uncovers the see-through window 4. The protective shield 1 is still in the lowered position. The protective shield 2 has a clear visor 5 that closes the see-through window 4 in the protective shield 2. Thus, a wearer's face is protected by the protective shield 2 and the clear visor 5 for example from particles ejected from a workplace the wearer faces toward. Although the clear visor 5 may not provide a sufficient protection against harmful light emitted from a welding arc in some cases, the clear visor 5 allows the wearer of the welding helmet 1 to see through the clear visor 5 at ambient light conditions. Therefore, in the lowered position of the protective shield 2 with the welding visor 3 being in the non-welding position the welding helmet 1 may for example be used for protecting a wearer during grinding or similar works.

As illustrated in FIG. 3 the welding helmet 1 is configured so that the welding visor 3 can be removed from the protective shield 2. The welding helmet 1 for this purpose has a bayonet coupling formed of a first bayonet part 7 at the welding visor 3 and a second bayonet part 8 at the protective shield 2 as described in more detail in the following.

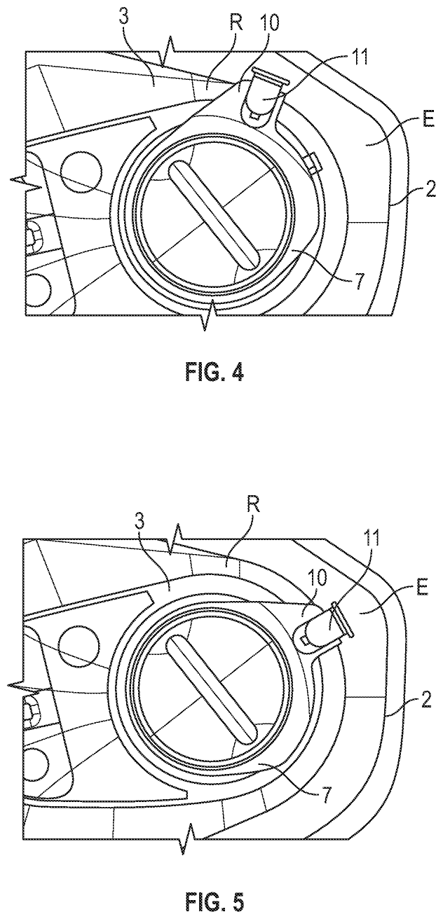

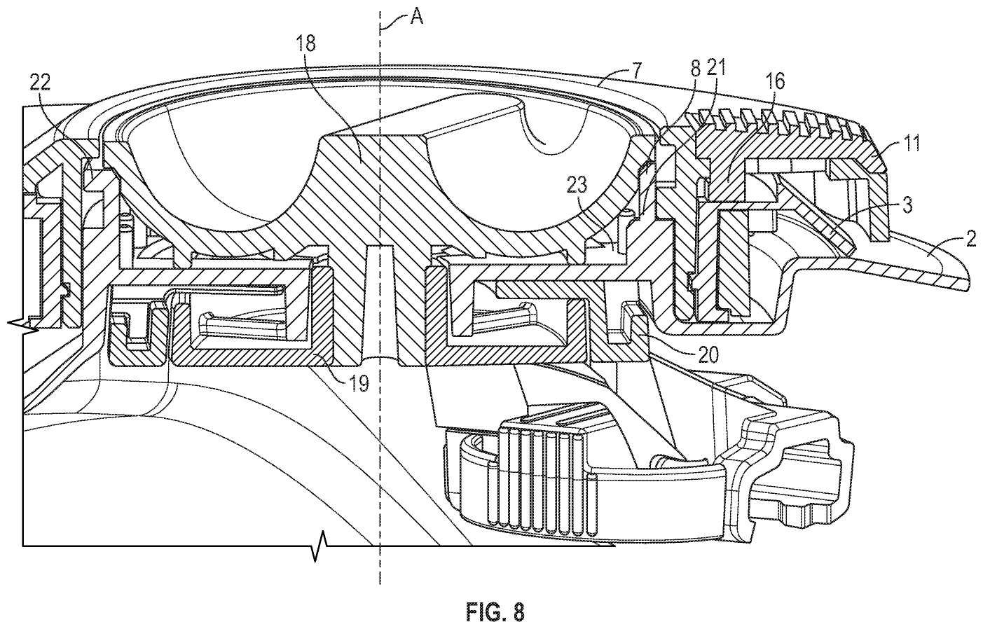

FIGS. 4, 5 and 6 illustrate the function of the bayonet coupling from an operation perspective, for example according to an operation performed by a wearer of the welding helmet 1. The first bayonet part 7 is rotatably connected to the welding visor 3. In particular, the first bayonet part 7 is rotatable between a release position R and an engage position E. The rotatability of the first bayonet part 7 is restricted to the rotation between the release position R and the engage position E. Therefore, the first bayonet part 7 is stopped in the release position R from a further rotation in a direction from the engage position E toward the release position R, and in the engage position E from a further rotation in a direction from the release position R toward the engage position E. The engage and release position are indicated in the Figures for explanation purposes and may not be particularly marked on the welding helmet 1. The release position R designates a position of the first bayonet part 7 in which the bayonet coupling can be disengaged. Further, the engage position E designates a position of the first bayonet part 7 in which a disengagement of the bayonet coupling is disabled. This means that in the engage position E the first and second bayonet part are rotatable relative to each other although they are axially engaged for preventing a disengagement. In this regard the term “axially” refers to a rotation axis A (see FIG. 8) of the rotatability of the first bayonet part 7.

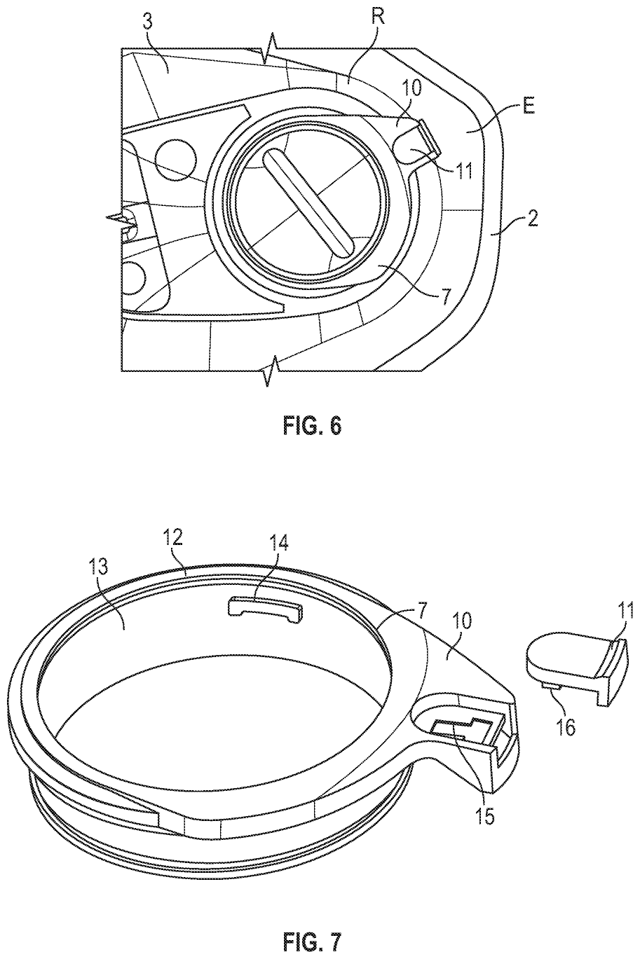

In the situation illustrated in FIG. 4 the first bayonet part 7 is positioned in the release position R. Accordingly the welding visor 3 is released so that it could be removed from the protective shield 2. In the situation illustrated in FIG. 5 the first bayonet part 7 is positioned in the engage position E, for example in consequence of turning the first bayonet part 7 from the release position R shown in FIG. 4 to the engage position E shown in FIG. 5.

As shown, the first bayonet part 7 has a lever 11 which carries a locking member 11. The locking member 11 is switchable between an unlocked position (shown in FIG. 5) and a locked position (shown in FIG. 6). In the unlocked position the rotation of the first bayonet part 7 from the engage position toward the release position is enabled. Therefore, in the unlocked position first bayonet part 7 can be rotated from the engage position toward the release position. However, in the locked position the rotation of the first bayonet part 7 from the engage position toward the release position is blocked. Therefore, in the locked position first bayonet part 7 cannot be rotated from the engage position toward the release position. The locking member in the locked position thus prevents the first bayonet part 7 to inadvertently move, for example in consequence of pivoting the welding visor 3 relative to the protective shield 2.

FIGS. 7, 8 and 9 illustrate the function and structure of the bayonet coupling in more detail. In FIG. 7 the first bayonet part 7 and the locking member 11 are shown in an exploded view. In particular the first bayonet part 7 is formed by a ring-shaped collar 12. The collar 12 has an inner cylindrical surface 13 from which a first engagement member 14 protrudes radially (of the cylinder axis of the cylindrical surface 13) inwardly. Although not visible in this view, the collar 12 has three first engagement members 14 that protrudes radially inwardly from the cylindrical surface 13. The three first engagement members 14 are uniformly distributed over the circumference of the inner cylindrical surface 13, in particular are angularly offset relative to each other at 120 degrees.

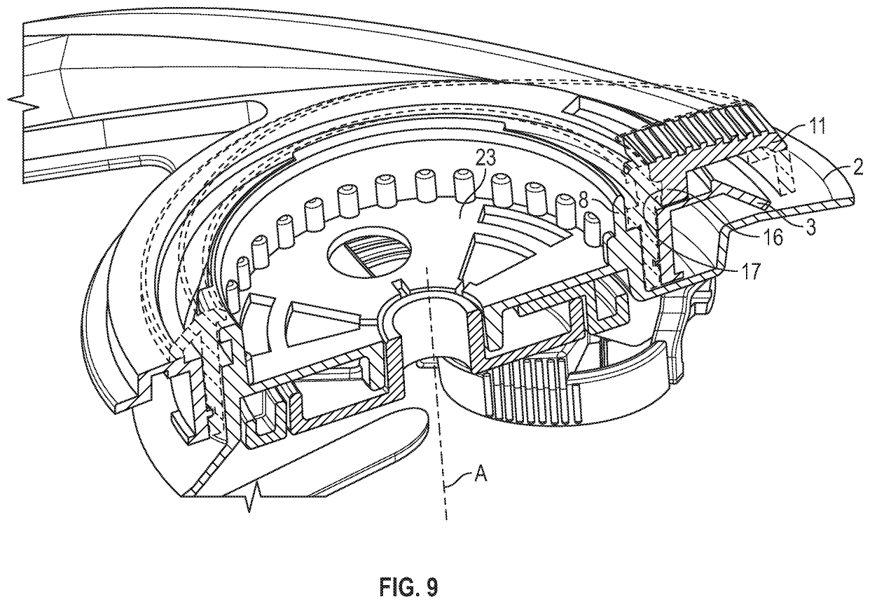

As shown, the lever 10 extends radially outwardly from the collar 12. The lever 10 has an oblong hole 15 within which a pin 16 is received (see FIG. 8) for guiding the locking member 11 during sliding between the unlocked position and the locked position. The welding visor 3 has a receptacle within which the pin 16 engages (FIG. 9) in the locked position. In the unlocked position the pin 16 and the receptacle 17 are disengaged (not shown) so that the first bayonet part 7 can be rotated toward the released position relative to the welding visor 3.

As shown in FIG. 8 the welding helmet 1 has a link that comprises a knob 18 that forms a screw bolt 18a, and a screw nut 19. The screw bolt 18a and the screw nut 19 are screwed with one another. The protective shield 2 is and a head suspension system 20 are connected by the link. In particular a portion of the protective shield 2 and a portion of the head suspension system 20 are arranged between the knob 18 and the screw nut 19. Thus, the protective shield 2 and the head suspension system 20 can be clamped together by the screw connection formed between the knob 18 and the screw nut 19. Thereby the protective shield 2 is also pivotally suspended at the head suspension system 20 by the link. In particular, the protective shield 2 may be pivoted relative to the head suspension system 20 about the link. Further, the knob 18 may be turned for tightening or loosening the screw connection formed by the link and thereby a retention (against pivoting) between the protective shield 2 and the head suspension system 3 may be adjusted.

The protective shield 2 further forms the second bayonet part 8. In the example the second bayonet part 8 is formed by a rim 21 that is connected to, in particular monolithically formed with, the protective shield 2. The second bayonet part 8 comprises a second engagement member 22 that protrudes radially outwardly from the rim 21. The second bayonet part 8 particularly comprises three second engagement members 22. The three second engagement members are uniformly distributed over the circumference of the inner cylindrical surface 13, in particular are angularly offset relative to each other at 120 degrees. Each of the first and second engagement members extend only partially circumferentially about the rotation axis A. Thus, the second engagement members 22 leave a space between each other through which the first engagement members 14 can pass when the first bayonet part 7 and the second bayonet part 8 are separated from each other or mated with each other.

In the example the rim 21 forms a recess 23 within which the knob 18 is rotatably accommodated.

1. A welding helmet, comprising:
a protective shield, and
a welding visor,
wherein the protective shield is suspended at a head suspension system, and the welding visor is detachably attachable to the protective shield by means of a bayonet coupling,
wherein the bayonet coupling comprises a first bayonet part and a second bayonet part, wherein the first bayonet part is rotatably connected to the welding visor for a rotation between a release position, in which the bayonet coupling is disengaged, and an engage position, in which a disengagement of the bayonet coupling is disabled, the bayonet coupling further forming a pivot for pivoting the welding visor relative to the protective shield between a welding position, in which the welding visor covers the see-through window, and a non-welding position, in which the welding visor uncovers the see-through window, and wherein a locking member is provided that is switchable between a locked position, in which the rotation of the first bayonet part from the engage position toward the release position is blocked, and an unlocked position, in which the rotation of the first bayonet part from the engage position toward the release position is enabled.

2. The welding helmet of claim 1, wherein the second bayonet part is formed by a rim that is connected to the protective shield.

3. The welding helmet of claim 2, wherein the first bayonet part is formed by a ring-shaped collar for mating on the rim, wherein the collar is rotatably mounted in a through-hole of the welding visor.

4. The welding helmet of claim 3, wherein the collar is axially fixed within the welding visor.

5. The welding helmet of claim 3, wherein the first bayonet part comprises a first engagement member that protrudes radially inwardly from the collar and wherein the second bayonet part comprises a second engagement member that protrudes radially outwardly from the rim, and wherein the engagement of the bayonet coupling is established by an engagement of the first and second engagement member.

6. The welding helmet of claim 3, wherein the first bayonet part has a lever that protrudes radially outwardly from the collar, wherein the locking member is displaceably arranged at the lever for a displacement between the locked and the unlocked position.

7. The welding helmet of claim 6, wherein the welding visor adjacent the through hole comprises a receptacle, and wherein the locking member in the locked position engages in the receptacle and thereby locks the first bayonet part and the welding visor with each other.

8. The welding helmet of claim 1, further having a link for pivotally suspending the protective shield at the head suspension system, wherein the link comprises a knob for adjusting a retention against pivoting of the protective shield relative to the head suspension system.

9. The welding helmet of claim 8, wherein the link further comprises a screw nut and wherein the knob comprises a screw bolt for establishing a screw connection with the screw nut, wherein the protective shield can be suspended at the head suspension system by establishing the screw connection with a portion of the protective shield and a portion of the head suspension system arranged between the knob and the screw nut.

10. The welding helmet of claim 1, wherein the see-through window comprises a clear protective plate.

11. The welding helmet of claim 1, wherein the welding visor comprises an Automatic Darkening Filter (ADF).

12. The welding helmet of claim 1, wherein in the welding position the welding visor seals an area on the protective shield in which the see-through window is located.

13. The welding helmet of claim 1, wherein the bayonet coupling in the engage position of the first bayonet part provides an engagement of the welding visor relative to the protective shield over the full pivot range between the welding visor and the protective shield.

14. The welding helmet of claim 13, wherein the pivot range between the welding visor and the protective shield between 50 degrees and 60 degrees.

15. The welding helmet of claim 1, wherein the second bayonet part is monolithically formed with the protective shield.
